(19) United States
(12) Patent Application Publication
(10) Pub. No.: US 2025/0283506 A1
(43) Pub. Date: Sep. 11, 2025

(54) MAGNETIC LEVITATION DEVICE AND AN ELECTROMAGNETIC ROTARY DRIVE

(71) Applicant: Levitronix GmbH, Zürich (CH)

(72) Inventor: Daniel STEINERT, Bülach (CH)

(21) Appl. No.: 19/048,716

(22) Filed: Feb. 7, 2025

(30) Foreign Application Priority Data
Mar. 5, 2024 (EP) 24161380.1

(51) Int. Cl. F16C 32/04

(52) U.S. Cl. CPC F16C 32/0478; F16C 2380/26

(57) ABSTRACT

A magnetic levitation device includes a stator having coil cores, each coil core made of sheet metal elements stacked in the circumferential direction of the stator. Each coil core has a first and lateral boundary surfaces, and each coil core comprises a longitudinal leg extending from a first end in an axial direction to a second end, and a transverse leg arranged at the second end of the longitudinal leg, and which extends in a radial direction. A concentrated winding is provided on each longitudinal leg, and surrounds the longitudinal leg. The stator has a cup-shaped recess into which the rotor can be inserted. The cup-shaped recess is arranged at an axial end of the stator. The transverse legs are arranged around the cup-shaped recess, and one of the first or the second lateral boundary surfaces has at least one slot.

### CROSS REFERENCE TO RELATED APPLICATIONS

This Applicant claims priority to European Application Number EP 24161380.1 filed Mar. 5, 2024, the contents of which are herein incorporated by reference.

### BACKGROUND

### Technical Field

The disclosure relates to a magnetic levitation device and to an electromagnetic rotary drive with such a magnetic levitation device.

### Background Information

Magnetic bearing devices for contactless magnetic bearing of a rotor have the advantage that they do not require mechanical bearings for the rotor. The rotor is supported or stabilized by magnetic forces which are generated by a stator of the magnetic bearing device. Due to the absence of mechanical bearings, such magnetic bearing devices are in particular suitable for pumping, mixing, centrifuging or stirring devices, with which very sensitive substances are conveyed, for example blood pumps, or on which very high demands are made with respect to purity, for example in the pharmaceutical industry or in the biotechnological industry, or with which abrasive or aggressive substances are conveyed, which would very quickly destroy mechanical bearings, for example pumps or mixers for slurry, sulfuric acid, phosphoric acid or other chemicals in the semiconductor industry.

In the biotechnology industry, such magnetic bearing devices are used, for example, in connection with bioreactors, e.g. in centrifugal pumps for conveying the fluids into or out of the bioreactor, or in mixing devices which mix the fluids in the bioreactor. In the semiconductor industry, such magnetic bearing devices are not only used for conveying aggressive or abrasive substances, but also, for example, for rotation devices with which wafers are rotated.

It is also known to use magnetic bearing devices for viscometers.

### SUMMARY

It has been determined that the temple construction is an advantageous magnetic bearing device design, to which the present disclosure also relates.

The characteristic feature of the temple construction is that the stator of the magnetic bearing device has a plurality of coil cores, each of which comprises a longitudinal leg extending from a first end in an axial direction to a second end. Here, the axial direction refers to that direction which is defined by the desired axis of rotation of the rotor, which is supported by the magnetic bearing device. The desired axis of rotation is that axis of rotation about which the rotor rotates in the operating state when it is in a centered and non-tilted position with respect to the stator. Each coil core comprises, in addition to the longitudinal leg, a transverse leg, also called a pole piece, which is arranged in each case at the second end of the longitudinal leg, and which extends in the radial direction-usually towards inside-, wherein the radial direction is perpendicular to the axial direction. Thus, the transverse leg extends substantially at a right angle to the longitudinal leg. The coil cores each have the shape of an L, wherein the transverse legs form the short legs of the L. The rotor to be supported is then arranged between the transverse legs.

The plurality of the longitudinal legs which extend in the axial direction, and which are reminiscent of the columns of a temple has given this construction its name.

In one design, the stator of the magnetic bearing device has, for example, six coil cores which are arranged circularly and equidistantly around a cup-shaped recess into which the rotor can be inserted. The first ends of the longitudinal legs are usually connected in the circumferential direction by a back iron, which serves to conduct the magnetic flux. The rotor to be supported comprises a magnetically effective core, for example a permanent magnetic disk or a permanent magnetic ring, which is arranged between the radially inside located ends of the transverse legs, and which rotates about the axial direction in the operating state, wherein the rotor is magnetically supported without contact with respect to the stator.

For such magnetic bearing devices, it is not necessarily the case that the magnetically effective core of the rotor must be designed in a permanent magnetic manner. There are also known such designs in which the magnetically effective core of the rotor is designed in a permanent magnetic-free manner, i.e., without permanent magnets. Then, the magnetically effective core of the rotor is, for example, designed in a ferromagnetic manner and is made, for example, of iron, nickel-iron, cobalt-iron, silicon iron, mu-metal, or another ferromagnetic material.

Furthermore, designs are possible in which the magnetically effective core of the rotor comprises both ferromagnetic materials and permanent magnetic materials. For example, permanent magnets can be placed or inserted into a ferromagnetic base body. Such designs are advantageous, for example, if one wishes to reduce the costs of large rotors by saving permanent magnetic material.

The longitudinal legs carry windings to generate the electromagnetic fields necessary for the contactless magnetic bearing of the rotor. For example, the windings are designed such that one concentrated winding is wound around each longitudinal leg, i.e., the coil axis of each concentrated winding extends in each case in the axial direction. Here, it is typical for the temple construction that the coil axes of the concentrated windings run in the axial direction and that the concentrated windings are not arranged in the radial plane in which the rotor or the magnetically effective core of the rotor is supported in the operating state.

Designs are possible in which exactly one concentrated winding is arranged on each longitudinal leg. In other designs, several, for example exactly two, concentrated windings are provided on each longitudinal leg. Designs are also possible in which windings are provided that are wound around two longitudinal legs that are adjacent in the circumferential direction, so that these two adjacent longitudinal legs are both located in the interior space of the concentrated winding.

The coil cores of the magnetic bearing devices known from the state of the art are usually designed in sheet metal. This means that several sheet metals in the shape of the coil cores are stacked in circumferential direction, insulated from each other. The sheet metal design of the coil cores prevents eddy currents for magnetic fields that run in the direction of the sheet metals, i.e. fields that follow the longitudinal leg in the axial direction and the transverse leg in the radial direction.

For magnetic fields that emerge laterally, i.e. in the circumferential direction, from the sheet metals of the longitudinal leg and the transverse leg, the insulation of the sheet metals is ineffective and thus eddy currents still occur, since these magnetic fields pass orthogonally through the sheet metals.

Particularly with magnetic bearing devices that have a large magnetic gap, wherein the magnetic gap is defined as the distance between the end face of the transverse leg and the magnetically effective core of the rotor in the radial direction, orthogonal field components cannot be neglected and generate significant eddy current losses.

In the context of this application, a large magnetic gap means a magnetic gap that is greater than 1% of the diameter of the magnetically effective core in the radial direction. In some cases, the magnetic gap can be greater than or equal to 5% of the diameter of the magnetically effective core in the radial direction.

It is therefore an object of the disclosure to propose a magnetic levitation device for contactless magnetic levitation of a rotor with a disk-shaped or ring-shaped magnetically effective core, which magnetic levitation device has lower eddy current losses than the previous state of the art.

Furthermore, it is an object of the disclosure to propose an electromagnetic rotary drive with such a magnetic levitation device.

The subject matter of the disclosure meeting this object is characterized by the features disclosed herein.

According to the disclosure, a magnetic levitation device is thus proposed for contactless magnetic levitation of a rotor, which comprises a disk-shaped or ring-shaped magnetically effective core, wherein the magnetic levitation device has a stator, which comprises a plurality of coil cores, wherein each coil core is made of elements in sheet metal, wherein the elements are stacked in the circumferential direction of the stator, wherein each coil core has a first lateral boundary surface and a second lateral boundary surface, wherein each coil core comprises a longitudinal leg, which extends from a first end in an axial direction to a second end, as well as a transverse leg, which is arranged at the second end of the longitudinal leg, and which extends in a radial direction, which is perpendicular to the axial direction, wherein at least one concentrated winding is provided on each longitudinal leg, which surrounds the respective longitudinal leg, wherein the stator further has a cup-shaped recess into which the rotor can be inserted, wherein the cup-shaped recess is arranged at an axial end of the stator, wherein the transverse legs are arranged around the cup-shaped recess, and wherein at least one of the first or the second lateral boundary surfaces has at least one slot.

The provision of slots in at least one of the two lateral boundary surfaces of the coil core provides an electrical insulation. This means that the at least one slot ensures that the path of the eddy currents in the coil core is interrupted and thus blocked. As a result, only small eddy currents remain in the coil core and the eddy current losses in the coil core are drastically reduced overall. Here, it is advantageous that the at least one slot should run parallel or at least approximately parallel to the course of the magnetic field in the coil core so as not to block it.

A number of different methods can be used to produce the slots. Inter alia, these include mechanical processes such as milling, punching or cutting, whereby the latter also includes the use of lasers or water jet cutters.

According to a preferred embodiment, the elements are made of electrical sheet metall. According to the general definition, an electrical sheet metal is understood to be a soft magnetic material for magnetic cores. There also exists the possibility of using mu-metal.

According to a preferred embodiment, the at least one slot extends only in the transverse leg. For applications in which the coil core must have a high stability, it can be advantageous that the at least one slot extends only in the transverse leg. Due to this embodiment, some eddy current losses are also reduced, since most of the effects that lead to eddy current losses occur in the area of the second end.

According to a preferred embodiment, the at least one slot extends in the transverse leg and in the longitudinal leg. This is advantageous in order to further reduce the eddy current losses in the coil core. It is variable how far the at least one slot extends in the longitudinal leg in axial direction in the direction of the first end of the longitudinal leg. From a small extension of 5% of the total length of the longitudinal leg in axial direction to an extension towards the first end of the longitudinal leg, all lengths of the at least one slot are possible.

In a preferred embodiment, the at least one slot has a rounding which redirects the slot from the radial direction to the axial direction.

According to a preferred embodiment, the at least one slot extends from the first lateral boundary surface to the second lateral boundary surface.

In a preferred embodiment, the extension of the at least one slot is shorter than the distance of the first lateral boundary surface from the second lateral boundary surface when viewed in the circumferential direction of the stator.

In other words, the at least one slot does not extend through the entire coil core in the circumferential direction of the stator. This means that the at least one slot is not provided in all elements of the sheet metal design of the coil core. This is advantageous because the majority of the eddy currents occur in the elements that are arranged directly or close to the two lateral boundary surfaces. Thus, the paths of the eddy currents in the coil core are interrupted by the at least one slot where they occur most frequently. This ensures a significant reduction in eddy current losses. Furthermore, this embodiment is advantageous for the stability of the coil core.

According to a preferred embodiment, several slots are provided which are arranged parallel or at least approximately parallel to each other.

The arrangement of the several slots parallel or at least approximately parallel to each other is advantageous, as they thus also run parallel or at least approximately parallel to the course of the magnetic field in the coil core, so that they do not block it.

Furthermore, it is preferred that the coil core has a rounding off at an axially upper end, which redirects the coil core from the axial direction to the radial direction.

For example, in the case of an L-shaped coil core, wherein the long part of the “L” is formed by the longitudinal leg and the short part of the “L” by the transverse leg, the radially outside located edge of the transverse leg, when viewed from the cup-shaped recess, which extends in the radial plane in the circumferential direction is designed in a rounded manner. This embodiment has the advantage that it has lower eddy current losses and is also easier to realize with regard to the construction.

In a preferred embodiment, a back iron is arranged at the first end of the longitudinal legs, which connects the first ends of all longitudinal legs, wherein the back iron is designed in a ring-shaped manner with a metallic strip which extends from a radially inner beginning to a radially outer end, wherein the strip forms several strip windings which lie flat against each other with respect to the radial direction.

According to a preferred embodiment, two concentrated windings are provided on each longitudinal leg, each of which surrounds the respective longitudinal leg, and which are arranged adjacent to one another with respect to the axial direction.

Furthermore, it is preferred that at least one slot is provided in each case both in the first lateral boundary surface and in the second lateral boundary surface.

This is advantageous because the majority of the eddy currents occur especially in the elements that are arranged directly at the two lateral boundary surfaces. Thus, the paths of the eddy currents in the coil core are interrupted by the at least one slot in each case where they occur most frequently. This ensures a significant reduction in eddy current losses.

According to a particularly preferred embodiment, the stator of the magnetic levitation device is designed to generate a torque with which the rotor can be magnetically driven without contact for rotation about the axial direction.

Here, the stator is designed as a bearing and drive stator, which is both the stator of the electric drive and the stator of the magnetic levitation. A magnetic rotating field can be generated with the electric windings of the stator, which, on the one hand, exerts a torque on the rotor, which causes its rotation about a desired axis of rotation and which, on the other hand, exerts an arbitrarily adjustable transverse force on the rotor, so that its radial position can be actively controlled or regulated.

Furthermore, an electromagnetic rotary drive which is designed as a temple motor is proposed by the disclosure, wherein the electromagnetic rotary drive comprises a magnetic levitation device according to the disclosure as well as a rotor with a disk-shaped or ring-shaped magnetically effective core, wherein the rotor can be inserted into the cup-shaped recess, and wherein the rotor is designed as the rotor of the electromagnetic rotary drive.

Such electromagnetic rotary drives are also known as bearingless motors. The term bearingless motor refers to an electromagnetic rotary drive in which the rotor is completely magnetically levitated with respect to the stator, wherein no separate magnetic bearings are provided.

Further advantageous measures and embodiments of the disclosure are apparent from the dependent claims.

### BRIEF DESCIPTION OF THE DRAWINGS

In the following, the disclosure will be explained in more detail with reference to embodiments and with reference to the drawing. In the drawing show:

FIG. 1 is a perspective view of a first embodiment of a magnetic levitation device according to the disclosure,

FIG. 2 is a perspective view of a single coil core of the magnetic levitation device from FIG. 1,

FIG. 3-FIG. 6 are different variants for the embodiment of a coil core, each in a perspective view,

FIG. 7 is a perspective view of a second embodiment of a magnetic levitation device according to the disclosure, and

FIG. 8 is a schematic sectional view for an embodiment of a stator housing.

### DETAILED DESCRIPTION

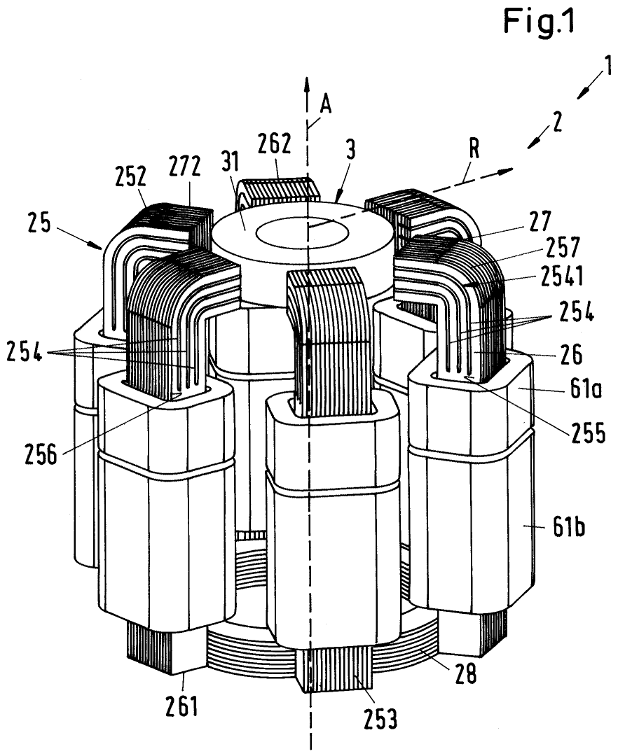

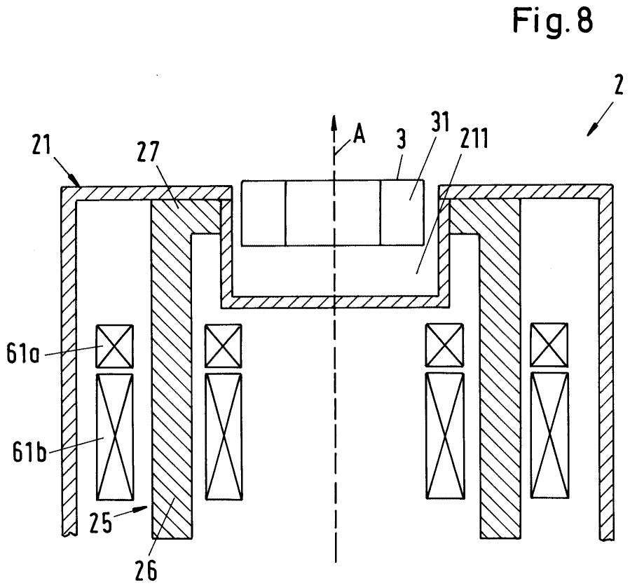

FIG. 1 shows a perspective view of an embodiment of a magnetic levitation device 1 according to the disclosure, which is designated in its entirety by the reference sign 1. The magnetic levitation device 1 is designed for the contactless magnetic levitation of a rotor 3, which comprises a disk-shaped or ring-shaped magnetically effective core 31. The magnetic levitation device 1 is designed according to the temple construction and comprises a stator 2. Normally, the stator 2 comprises a stator housing 21 (FIG. 8), which however is not represented in FIG. 1 for reasons of a better overview. For this reason, FIG. 8 illustrates an embodiment of a stator housing 21 in a schematic sectional view.

A cup-shaped recess 211 is provided on one axial end of the stator housing 21 into which the rotor 3 can be inserted. The rotor 3 is designed for rotation about a desired axis of rotation. This desired axis of rotation defines an axial direction A. Normally, the central axis of the stator 2, which extends in the axial direction A, coincides with the desired axis of rotation. The desired axis of rotation designates that axis about which the rotor 3 rotates in the operating state when the rotor 3 is in a centered and non-tilted position with respect to the stator 2, as represented in FIG. 1.

The stator 2 has a plurality of coil cores 25-here six coil cores 25, wherein each coil core 25 is made of elements 253 in sheet metal. The elements 253 are stacked in the circumferential direction of the stator 2 and each coil core 25 has a first lateral boundary surface 255 and a second lateral boundary surface 256. The circumferential direction refers to that direction which stands perpendicular on the radial direction R and perpendicular on the axial direction A. Furthermore, each coil core 25 comprises a longitudinal leg 26, which extends from a first end 261 in an axial direction A to a second end 262, and a transverse leg 27, which is arranged at the second end 262 of the longitudinal leg and extends in a radial direction which is perpendicular to the axial direction A.

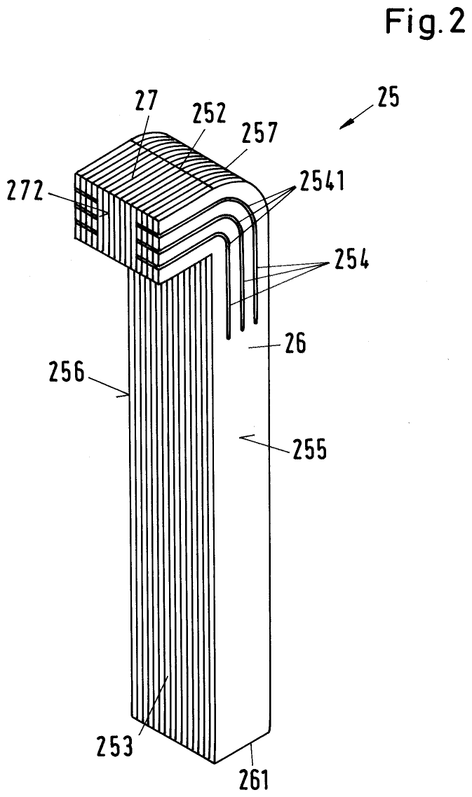

For better understanding, a perspective view of a single coil core 25 of the magnetic levitation device 1 from FIG. 1 is represented in FIG. 2.

The coil cores 25 of the stator 2 are arranged equidistantly on a circular line so that the transverse legs 27 surround the magnetically effective core 31 of the rotor 3 when the rotor 3 is inserted into the cup-shaped recess 211.

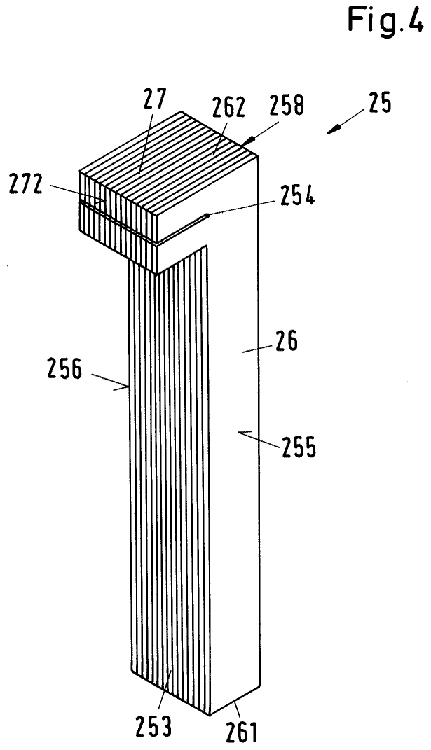

In this embodiment, the coil cores 25 have a rounding off 257 at an axially upper end 252, which redirects the coil core 25 from the axial direction A to the radial direction R. The coil cores 25 have a rounding off 257 of the outer edge 258 (FIG. 4) of the coil core 25 at the axially upper end of the coil core 252. This means that in this embodiment the radially outside located edge 258 at the axially upper end 252 of the coil core 25 is a broken edge or a rounded edge, as represented by the rounding off 257. Depending on the embodiment of the coil core 25, the rounding off 257 can extend only in areas of the longitudinal leg 26 or only in areas of the transverse leg 27 or both in areas of the longitudinal leg 26 and of the transverse leg 27. This has the advantage that this embodiment of the coil core 25 has lower eddy current losses and is also easier to realize with regard to the construction.

At least one concentrated winding 61 is arranged at each longitudinal leg 26, which surrounds the respective longitudinal leg 26. In other embodiments, more than one concentrated winding 61 can also be arranged at the longitudinal legs 26. For example, there are embodiments, as represented here in FIG. 1, wherein exactly two concentrated windings 61a, 61b are provided in each case on each of the longitudinal legs 26, each of which surrounds the respective longitudinal leg 26, wherein the two windings 61a, 61b arranged on the same longitudinal leg 26 are arranged adjacent to each other with respect to the axial direction A.

The concentrated windings 61a, 61b serve to generate electromagnetic fields with which the rotor 3 can be magnetically levitated without contact in the cup-shaped recess 211 (FIG. 8).

The elements 253 can be made of electrical sheet metall. According to the general definition, an electrical sheet metal is understood to be a soft magnetic material for magnetic cores. There also exists the possibility of using mu-metal for the strip.

The number of elements 253 in all embodiments and figures is to be understood as purely exemplary. The number can be larger or smaller than represented.

In the first embodiment of the magnetic levitation device 1 according to the disclosure represented in FIG. 1, three slots 254 are arranged in each case both in the first and the second lateral boundary surfaces 255, 256. The slots 254 extend both in the transverse leg 27 and in the longitudinal leg 26. The insertion of the slots 254 in the two lateral boundary surfaces 255, 256 of the coil core 25 provides an electrical insulation. This means that the slots 254 ensure that the path of the eddy currents in the coil core 25 is interrupted and thus blocked. As a result, only small eddy currents remain in the coil core 25 and the eddy current losses in the coil core 25 are drastically reduced overall.

In this embodiment, each of the slots 254 has a rounding 2541 which redirects the respective slot 254 from the radial direction R to the axial direction A. The slots 254 are arranged parallel or at least approximately parallel to each other and parallel or at least approximately parallel to the course of the magnetic field in the coil core 25. This has the advantage that the slots 254 thus do not obstruct and/or block the course of the magnetic field in the coil core 25.

Here, the slots 254 do not extend over the entire extension of the longitudinal leg 26 in axial direction A, but only in part and end before an axially upper end of the concentrated winding 61a.

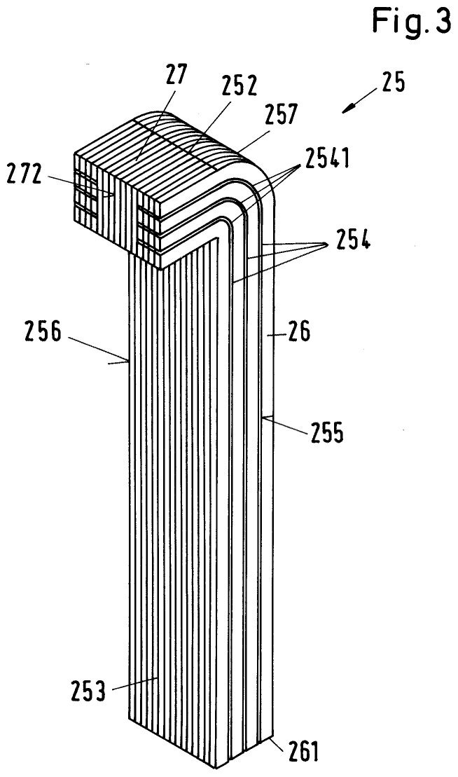

Furthermore, however, embodiments are also possible in which the at least one slot 254 has a longer extension in the longitudinal leg 26 than in the embodiment represented in FIG. 2. Such a possible embodiment is represented in FIG. 3. The perspective view there of a variant for the embodiment of a coil core 25 shows the maximum possible extension of a slot 254 in a coil core 25.

Of course, it is possible that the slots 254 can have any extension length in the longitudinal leg 26. It is also possible that the slots 254 also extend only in the transverse leg 27.

In this embodiment, the extension of the slots 254, when viewed in the circumferential direction of the stator 2, is shorter than the distance between the first lateral boundary surface 255 and the second lateral boundary surface 256. In other words, the slots 254 do not penetrate all elements 253 of the coil cores 25, but only a certain number. In the present embodiment, there are eight elements 253 per coil core 25, i.e. four elements 253 in each case when viewed from each lateral boundary surface 255, 256.

This is advantageous because the majority of the eddy currents occur especially in the elements 253, which are arranged directly on or close to the two lateral boundary surfaces 255, 256. Thus, the paths of the eddy currents in the coil core 25 are interrupted by the slot 254 where they occur most frequently. This ensures a significant reduction in eddy current losses. Furthermore, an incomplete penetration of the slots 254 through all elements 253 of the coil core 25 is advantageous for the stability of the coil core 25.

However, embodiments are also possible in which the slots 254 extend from the first lateral boundary surface 255 to the second lateral boundary surface 256.

According to a particularly preferred embodiment, the stator 2 is designed such that, in addition to the contactless magnetic levitation of the rotor 3, it can also exert a torque on the rotor 3 or the magnetically effective core 31 of the rotor 3, which drives the rotor 3 for rotation about the desired axis of rotation. This means that in this preferred embodiment the rotor 3 can be driven for rotation about the axial direction A.

In this embodiment, the concentrated windings 61 thus generate electromagnetic rotating fields with which the rotor 3 can be both magnetically levitated without contact with respect to the stator 2 and can also be driven without contact for rotation about the axial direction A.

It is understood that the number of six coil cores 25, although preferred, is only to be understood as an example. Of course, such embodiments are also possible in which the stator 2 has fewer than six, e.g. five or four or three coil cores 25, or such embodiments in which the stator 2 has more than six, e.g. seven or eight or nine or twelve coil cores 25 or any larger number of coil cores 25.

The rotor 3 comprises the magnetically effective core 31, which is designed in a ring-shaped or disk-shaped manner. According to the representation in FIG. 1, the magnetically effective core 31 is designed as a ring and defines a magnetic center plane. Alternatively, the magnetically effective core 31 can also be designed as a disk. Normally, in the case of a disk-shaped or ring-shaped magnetically effective core 31, the magnetic center plane is the geometric center plane of the magnetically effective core 31 of the rotor 3, which is perpendicular to the axial direction A. In the operating state, the magnetically effective core 31 is levitated in a radial plane, which stands perpendicular on the axial direction A.

The radial plane is indicated in FIG. 1 by the line of a radial direction R, which stands perpendicular on the axial direction A. The radial plane is that plane which stands perpendicular on the axial direction A and contains a radial direction R. The radial plane is that plane in which the magnetically effective core 31 of the rotor 3 is actively magnetically levitated between the end faces 272 in the stator 2 in the operating state. If the rotor 3 is not tilted and is not deflected in axial direction A, the magnetic center plane lies in the radial plane E. The radial plane defines the x-y plane of a Cartesian coordinate system whose z-axis runs in axial direction A.

The radial position of the magnetically effective core 31 or the rotor 3 refers to the position of the rotor 3 in the radial plane E.

Since it is sufficient for the understanding of the disclosure, only the magnetically effective core 31 of the rotor 3 is represented in FIG. 1. It is understood that the rotor 3 can of course also comprise further components such as jackets or encapsulations, which are preferably made of a plastic, or of a metal or of a metal alloy or of a ceramic or a ceramic material. Furthermore, the rotor 3 can also comprise vanes for mixing, stirring or pumping fluids or other components.

When the rotor 3 is inserted into the cup-shaped recess 211 (FIG. 8), the rotor 3 and in particular the magnetically effective core 31 of the rotor 3 is surrounded by the radially outwardly arranged end faces 272 of the transverse legs 27 of the coil cores 25 of the stator 2. Thus, the transverse legs 27 form a plurality of pronounced stator poles—in this case six stator poles.

When the magnetically effective core 31 of the rotor 3 is in its desired position during operation, the magnetically effective core 31 is centered between the end faces 272 of the transverse legs 27. According to the representation, the concentrated windings 61 are arranged below the radial plane and are aligned such that their coil axes extend in axial direction A.

All first ends 261 of the longitudinal legs 26—i.e., the lower ends 261 according to the representation (FIG. 1)—are connected to each other by a back iron 28. The back iron 28 is preferably designed in a ring-shaped manner. Such embodiments are possible (see FIG. 1, for example) in which the back iron 28 extends radially inwardly along all first ends 261 of the longitudinal legs 26.

In order to generate the electromagnetic fields required for the magnetic levitation of the rotor 3 and optionally for the generation of a torque on the rotor 3, the longitudinal legs 26 of the coil cores 25 carry the windings designed as concentrated windings 61.

In the operating state, those electromagnetic rotating fields are generated with these concentrated windings 61 with which an arbitrarily adjustable transverse force in the radial direction can be exerted on the rotor 3, so that the radial position of the rotor 3, i.e. its position in the radial plane perpendicular to the axial direction A, can be actively controlled or regulated. Optionally, a torque is additionally effected on the rotor 3 with these electromagnetic rotating fields.

The “magnetically effective core 31” of the rotor 3 refers to that region of the rotor 3 which magnetically cooperates with the stator 2 for the generation of magnetic levitation forces and optionally for torque generation.

As already mentioned, the magnetically effective core 31 is designed in a ring-shaped manner in this embodiment. Furthermore, the magnetically effective core 31 is designed in a permanent magnetic manner. For this purpose, the magnetically effective core 31 can comprise at least one permanent magnet, but also several permanent magnets, or—as in the embodiment described here—consist entirely of a permanent magnetic material, so that the magnetically effective core 31 is the permanent magnet. For example, the magnetically effective core 31 is magnetized in the radial direction.

Those ferromagnetic or ferrimagnetic materials, which are magnetically hard, that is which have a high coercive field strength, are typically called permanent magnets. The coercive field strength is that magnetic field strength which is required to demagnetize a material. Within the framework of this application, a permanent magnet is understood as a component or a material, which has a coercive field strength, more precisely a coercive field strength of the magnetic polarization, which amounts to more than 10'000 A/m.

Such embodiments are also possible in which the magnetically effective core 31 is designed in a permanent magnet-free manner, i.e., without permanent magnets. The rotor 3 is then designed, for example, as a reluctance rotor. Then, the magnetically effective core 31 of the rotor 3 is made of a soft magnetic material, for example. Suitable soft magnetic materials for the magnetically effective core 31 are, for example, ferromagnetic or ferrimagnetic materials, i.e., in particular iron, nickel-iron, cobalt-iron, silicon iron, mu-metal.

Furthermore, embodiments are possible in which the magnetically effective core 31 of the rotor 3 comprises both ferromagnetic materials and permanent magnetic materials. For example, permanent magnets can be placed or inserted into a ferromagnetic base body. Such embodiments are advantageous, for example, if one wishes to reduce the costs of large rotors by saving permanent magnetic material.

Embodiments are also possible in which the rotor is designed according to the principle of a cage rotor.

The ring-shaped back iron 28 can be made of a soft magnetic material because it serves as flux conducting element to conduct the magnetic flux. It is also possible that the coil cores 25 of the stator 2 are also made of a soft magnetic material.

Suitable soft magnetic materials for the coil cores 25 and the back iron 28 are, for example, ferromagnetic or ferrimagnetic materials, i.e., in particular iron, nickel-iron, cobalt-iron, silicon-iron or mu-metal. In this case, for the stator 2, a design as a stator sheet metal stack is preferred, in which the back iron 28 is designed in sheet metal, i.e., it consists of several thin sheet metal elements, which are stacked parallel to each other in axial direction A. All flux conducting elements are identically designed, i.e., in this case substantially ring-shaped and also with the same thickness in each case. Thus, the back iron 28 itself is substantially designed in a ring-shaped manner and extends radially inwardly along the first ends 261 of the longitudinal legs 26 in the assembled state.

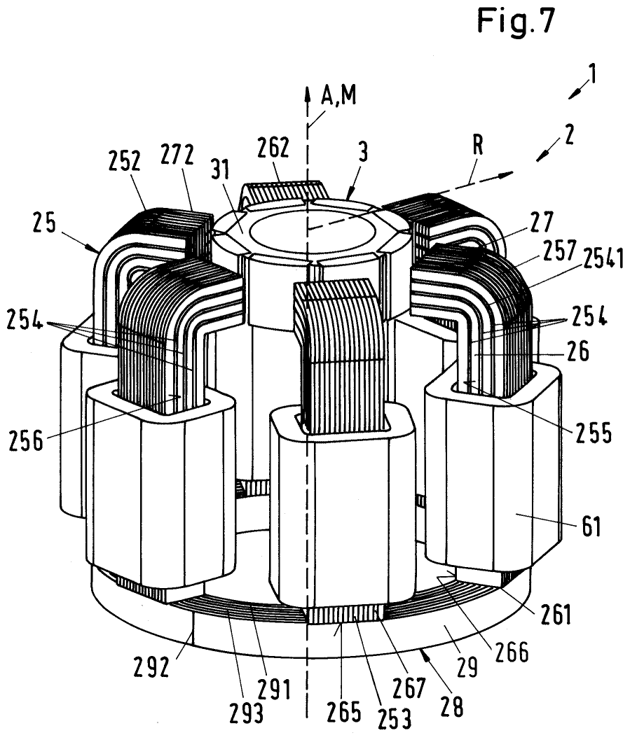

Embodiments in which a so-called tape wound toroidal core is used as the back iron 28 are also possible. This is a coiled strip 29. Such a design is realized in the second embodiment, which is represented in FIG. 7.

Furthermore, it is possible that the back iron 28 consists of pressed and subsequently sintered grains of the aforementioned materials. The metallic grains are preferably embedded in a plastic matrix so that they are at least partially insulated from each other, whereby eddy current losses can be minimized. Thus, soft magnetic composites, which consist of electrically insulated and compressed metal particles are also suitable for the stator. In particular, these soft magnetic composites, which are also designated as SMC (Soft Magnetic Composites), can consist of iron powder particles which are coated with an electrically insulating layer. These SMC are then formed into the desired shape by powder metallurgy processes.

During operation of the magnetic levitation device 1, the magnetically effective core 31 of the rotor 3 cooperates with the stator 2 in such a way that the rotor 3 can be magnetically levitated without contact with respect to the stator 2 and preferably can also be magnetically set in rotation without contact about the axial direction A. In this case, it is particularly advantageous that the same windings 61, with which the magnetic levitation of the rotor 3 is effected, also serve to generate a torque on the rotor 3. Preferably, three degrees of freedom of the rotor 3 can then be actively regulated, namely its position in the radial plane E and its rotation. With respect to its axial deflection from the radial plane E in axial direction A, the magnetically effective core 31 of the rotor 3 is passively magnetically stabilized by reluctance forces, i.e., it cannot be controlled. The magnetically effective core 31 of the rotor 3 is also passively magnetically stabilized with respect to the remaining two degrees of freedom, namely tilting with respect to the radial plane perpendicular to the desired axis of rotation. By the cooperation of the magnetically effective core 31 with the coil cores 25, the rotor 3 is thus passively magnetically levitated or passively magnetically stabilized in axial direction A and against tilting (a total of three degrees of freedom) and actively magnetically levitated in the radial plane (two degrees of freedom).

As is generally the case, an active magnetic levitation is also referred to in the framework of this application as one which can be actively controlled or regulated, for example by the electromagnetic fields generated by the concentrated windings 61. A passive magnetic levitation or a passive magnetic stabilization is one that cannot be controlled or regulated. The passive magnetic levitation or stabilization is based, for example, on reluctance forces, which bring the rotor 3 back again to its desired position when it is deflected from its desired position, i.e., for example, when it is displaced or deflected in axial direction A or when it is tilted.

In the magnetic levitation device 1, in contrast to classic magnetic bearings, the magnetic levitation—and optionally the generation of a torque acting on the rotor—is realized by electromagnetic rotating fields. For the combined generation of the magnetic levitation forces and a torque for rotating the rotor 3 about the axial direction A, it is possible on the one hand—as shown in FIG. 7—to arrange exactly one concentrated winding 61 at each longitudinal leg 26.

On the other hand, embodiments are also possible in which two different winding systems are provided for the combined generation of the magnetic levitation forces and a torque for rotating the rotor 3. For this purpose, for example, exactly two concentrated windings 61a, 61b are arranged in each case at each longitudinal leg 26, as represented in FIG. 1, which are arranged adjacent to each other with respect to the axial direction A. One of these two windings 61a, 61b belongs to the first of the two winding systems and the other to the second of the two winding systems.

In the embodiment represented in FIG. 7 with exactly one concentrated winding 61 at each coil core 25, the values for the current required for the levitation and the current required for the generation of the torque determined in each case, for example, in the control unit are added or superimposed by calculation—e.g., with the aid of software. Then, the resulting total current is impressed into the respective concentrated winding 61.

If the stator 2 of the magnetic levitation device 1 according to the disclosure is designed for generating a torque, the magnetic levitation device 1 is suitable for an electromagnetic rotary drive, which is designed as a temple motor. It is also possible that the magnetic levitation device 1 according to the disclosure is also suitable for other devices, such as centrifugal pumps, mixing devices for mixing flowable substances, stirring devices, for example for mixing a fluid in a tank, fans or also for devices for supporting and rotating wafers, for example in semiconductor production.

FIG. 4 shows a perspective view of a further variant for the embodiment of a coil core 25. In this variant, the coil core 25 is not rounded at the edge 258, but is designed in a rectangular manner. In this embodiment, only one slot 254 extends in the transverse leg 27, there is no extension of the slot 254 in the longitudinal leg 26. The slot 254 extends from the first lateral boundary surface 255 to the second lateral boundary surface 256, i.e. it penetrates all elements 253 of the coil core 25.

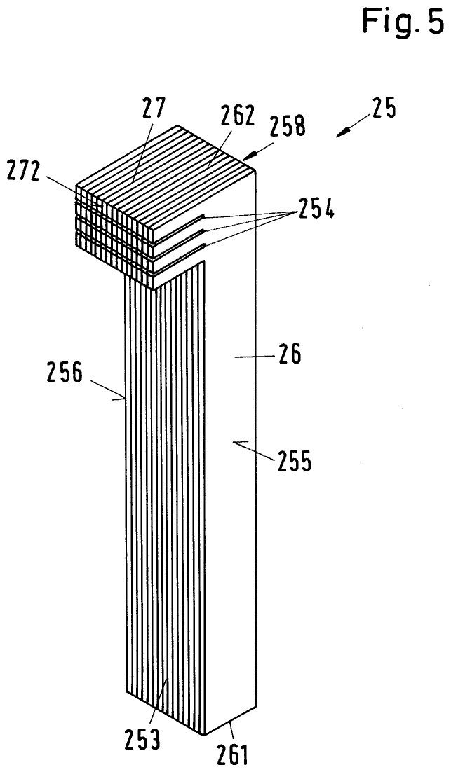

FIG. 5 shows a perspective view of a further variant for the embodiment of a coil core 25. One difference to the variant in FIG. 4 is that three slots 254 are present in this variant, all of which extend from the first lateral boundary surface 255 to the second lateral boundary surface 256.

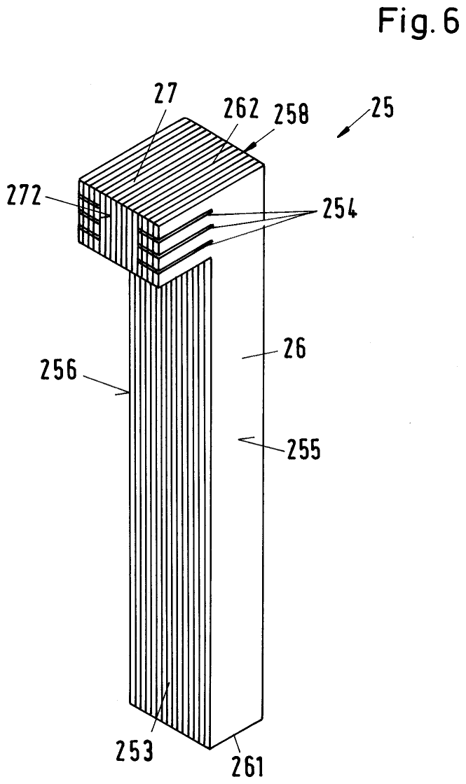

FIG. 6 shows a perspective view of a further variant for the embodiment of a coil core 25. Here, the difference to the variant from FIG. 4 is, on the one hand, that three slots 254 are present and, on the other hand, that the extension of the three slots 254 is shorter than the distance between the first lateral boundary surface and the second lateral boundary surface when viewed in the circumferential direction of the stator 2. This means that the slots 254 do not penetrate all the elements 253 of the coil cores 25, but only a certain number. In the present embodiment, there are eight elements 253, i.e. four elements 253 each, when viewed from each lateral boundary surface 255, 256.

Of course, the described variants and embodiments of the coil cores 25 from the FIGS. 2-6 can also be combined with each other in any form. All explanations also apply in the same way or analogously same to all variants.

FIG. 7 shows a perspective view of a second embodiment of a magnetic levitation device 1 according to the disclosure. In the following description of the second embodiment of the magnetic levitation device 1, only the differences to the first embodiment from FIG. 1 are explained in more detail. The explanations of the first embodiment also apply in the same way or analogously to the second embodiment. The same reference signs designate the same features that were explained with reference to the first embodiment or features equivalent in function.

One difference between this embodiment of a magnetic levitation device 1 and the embodiment from FIG. 1 is that the back iron 28 is designed differently. The back iron 28 is designed in a ring-shaped manner with a metallic strip 29, which extends from a radially inner beginning 291 to a radially outer end 292. The strip 29 forms several strip windings 293, which lie flat against each other with respect to the radial direction R. The longitudinal legs 26 are delimited at the first end 261 by an axial end face 265, against which the back iron 28 rests. The back iron 28 forms a circular ring, the radial width of which is equal to the radial width of the end faces 265 of the longitudinal legs 26. This means that the radially inner beginning 291 is flush in axial direction A with a radially inside located inner surface 266 of the longitudinal leg 26 and the radially outer end 292 is flush in axial direction A with a radially outside located outer surface 267 of the longitudinal leg 26.

Inter alia, this has the advantage that there is more space inside the stator 2, which can be used to install other components in it. In doing so, the stator 2 together with the stator housing 21 can be made smaller and more compact, which increases the flexibility of use of the magnetic levitation device 1.

A further advantage of such an arrangement of the back iron 28 results from the arrangement of the strip windings 293 of the strip 29 of the tape wound toroidal core. Due to the fact that the strip windings 293 are arranged perpendicular on the radial direction R, they have an orientation that is parallel to the magnetic field course in the longitudinal legs 26. As a result, the magnetic field from the longitudinal legs 26 enters the back iron 28 in the axial direction A and thus parallel to the strip windings 293. This means that the magnetic field does not penetrate any of the strip windings 293 in the radial direction R, whereby eddy current losses are avoided.

A further difference to the embodiment in FIG. 1 is that the extension of the slots 254 in the longitudinal leg 26 is longer. Depending on the field of application of the magnetic levitation device 1, this can be advantageous in order to achieve a greater reduction in eddy current losses.

Furthermore, the rotor 3 in this embodiment differs from that in the embodiment in FIG. 1. The rotor 3 used here is designed as a so-called four-pole-pair rotor.

However, it is of course also possible to operate the magnetic levitation device 1 shown in this embodiment with any other rotors 3. Some of these have already been explained in previous sections.

Needless to say, that all embodiments with their respective characteristics shown in the descriptions of the figures can be combined with each other in any way.

Furthermore, it is possible that all of the shown embodiments of a coil core 25 can be designed in such a way that the space available to the rotor 3 in the magnetic levitation device 1 is increased. This is achieved by a special external shape of the coil cores 25.

In the process, the coil core 25 is divided into an axially lower section and an axially upper section, wherein the lower section and the upper section are arranged adjacent to each other with respect to the axial direction A. The transverse leg 27 is arranged at the axially upper section. For each coil core 25, the end face 272 of the transverse leg 27 has a first distance in radial direction from the axially lower section of the associated longitudinal leg 26, and a second distance in radial direction from the axially upper section, wherein the second distance is greater than the first distance. This means that each longitudinal leg 26 is designed in such a way that the axially upper section is displaced outwards in radial direction with respect to the axially lower section, so that the space available for the rotor 3 between the end faces 272 increases without the risk of a direct transfer of the magnetic flux between the longitudinal leg 26 and the magnetically effective core 31 of the rotor 3 existing. Due to the fact that the axially upper sections are offset radially outwards with respect to the radial direction and relative to the axially lower sections, the distance, namely the second distance, between the longitudinal legs 26 and the end faces 272 increases in the area of the axially upper sections. In doing so, the distance between the magnetically effective core 31 of the rotor and the longitudinal legs 26, particularly in the area of the axially upper sections, also increases.

Such coil cores 25 just described are designed analogously to those illustrated in FIG. 3 in the European patent application EP4084304A1.

FIG. 8 shows a schematic sectional view for an embodiment of a stator housing 21. In the embodiments in FIG. 1 and FIG. 7, this stator housing 21 is not represented for reasons of a better overview. FIG. 8 is only intended to serve as an illustration to show how an encapsulation of the interior of the stator 2 necessary for the operation of the magnetic levitation device 1 looks. For this reason, the other components of the stator 2 are only represented schematically and are to be understood as purely illustrative.

Embodiments of the stator housing 21 are also possible in which the cup-shaped recess 211 merges into a bore that extends centrally along the central axis of the stator 2 in axial direction A through the entire stator housing 21.

During operation of the magnetic levitation device 1 in fields in which, for example, chemically aggressive substances are used, it is important that the interior of the stator 2 is securely encapsulated and thus protected from these substances. To ensure that a rotor 3 can still be used, the stator housing 21 has a cup-shaped recess 211 into which the rotor 3 can be inserted.

What is claimed is:

1. A magnetic levitation device for contactless magnetic levitation of a rotor, which comprises a disk-shaped or ring-shaped magnetically effective core, the magnetic levitation device comprising:
a stator comprising a plurality of coil cores, each coil core of the plurality of coil cores made of sheet metal elements, the sheet metal elements being stacked in a circumferential direction of the stator, each coil core of the plurality of coil cores having a first lateral boundary surface and a second lateral boundary surface, each coil core of the plurality of coil cores comprising a longitudinal leg extending from a first end in an axial direction to a second end, and a transverse leg arranged at the second end, and extending in a radial direction, the radial direction being perpendicular to the axial direction, at least one concentrated winding is provided on each longitudinal leg, and surrounding a respective longitudinal leg, the stator including a cup-shaped recess into which the rotor is capable of being inserted, the cup-shaped recess arranged at an axial end of the stator, and the transverse legs of the plurality of coil cords are arranged around the cup-shaped recess, at least one of the first or the second lateral boundary surfaces having at least one slot.

2. The magnetic levitation device according to claim 1, wherein the sheet metal elements are made of electrical sheet metal.

3. The magnetic levitation device according to claim 1, wherein for each coil cord of the plurality of coil cords, the at least one slot extends only in the transverse leg.

4. The magnetic levitation device according to claim 1, wherein for each coil cord of the plurality of coil cords, the at least one slot extends in the transverse leg and in the longitudinal leg.

5. The magnetic levitation device according to claim 4, wherein for each coil cord of the plurality of coil cords, the at least one slot has a rounding redirecting the slot from the radial direction to the axial direction.

6. The magnetic levitation device according to claim 1, wherein for each coil cord of the plurality of coil cords, the at least one slot extends from the first lateral boundary surface to the second lateral boundary surface.

7. The magnetic levitation device according to claim 1, wherein an extension of the at least one slot is shorter than a distance of the first lateral boundary surface from the second lateral boundary surface when viewed in the circumferential direction of the stator,

8. The magnetic levitation device according to claim 1, wherein the at least one slot includes several slots arranged parallel or at least approximately parallel to each other.

9. The magnetic levitation device according to claim 1, wherein each coil core of the plurality of coil cords has a rounding off at an axially upper end, the rounding off redirecting the coil core from the axial direction to the radial direction.

10. The magnetic levitation device according to claim 1, wherein a back iron is arranged at the first end of each of the longitudinal legs of the plurality of coil cords and connects the first ends of the longitudinal legs, the back iron is ring-shaped with a metallic strip extending from a radially inner beginning to a radially outer end, and the strip forms several strip windings which lie flat against one another with respect to the radial direction.

11. The magnetic levitation device according to claim 1, wherein the at least one concentrated winding includes two concentrated windings provided on each longitudinal leg of the plurality of coil cords, each of the two concentrated windings surrounds a respective longitudinal leg, and are arranged adjacent to one another with respect to the axial direction.

12. The magnetic levitation device according to claim 1, wherein at least one slot is provided in the first lateral boundary surface and in the second lateral boundary surface.

13. The magnetic levitation device according to claim 1, wherein the rotor is capable of being driven magnetically without contact for rotation about the axial direction by a torque generated by the stator.

14. An electromagnetic rotary drive, which is a temple motor, the electromagnetic rotary drive comprising:
a magnetic levitation device according to claim 13; and
a rotor with the disk-shaped or ring-shaped magnetically effective core, the rotor configured to be inserted into the cup-shaped recess, and is the rotor of the electromagnetic rotary drive.
